(19) United States
(12) Patent Application Publication
(10) Pub. No.: US 2026/0091370 A1
(43) Pub. Date: Apr. 2, 2026

(54) CATALYST FOR EPOXIDATION REACTIONS AND PREPARATION THEREOF

(71) Applicants: Evonik Operations GmbH, Essen (DE); REPSOL, S.A., Madrid (ES)

(72) Inventors: Nicole WILDE, Rodgau (DE); Dorit WOLF, Oberursel (DE); Heiko MORELL, Rodgau (DE); Christian PANZ, Wesseling-Berzdorf (DE); Sven MÜLLER, Bonn (DE); Jordi PEDROLA VIDAL, MÓSTOLES (ES); Marcio Rafael CORDEIRO ALVES DO ROSARIO, MÓSTOLES (ES); Luis VEGA BERMEJO, MÓSTOLES (ES); Raquel MARTINEZ SILVESTRE, MÓSTOLES (ES)

(21) Appl. No.: 19/110,128

(22) PCT Filed: Aug. 29, 2023
(86) PCT No.: PCT/EP2023/073670

§ 371 Date: Mar. 10, 2025

(30) Foreign Application Priority Data
Sep. 12, 2022 (EP) 22195136.1

(51) Int. Cl. B01J 21/06; B01J 21/08; B01J 35/61; B01J 35/63; B01J 35/64; B01J 37/02; B01J 37/06; B01J 37/08; C07D 303/04

(52) U.S. Cl. CPC B01J 21/063; B01J 21/08; B01J 35/615; B01J 35/617; B01J 35/633; B01J 35/635; B01J 35/647; B01J 37/0236; B01J 37/06; B01J 37/08; C07D 303/04

(57) ABSTRACT

The present invention relates to processes for preparing propylene oxide, comprising reaction of propene with ethylbenzene hydroperoxide in the presence of a catalyst, as well as to catalysts employed in such processes and to methods for their manufacture. The catalysts of the invention are prepared in a process comprising sol-gel-synthesis of catalyst hydrogel-precursor, drying of catalyst hydrogel precursor, calcining of dried catalyst hydrogel-precursor, and optionally hydrophobizing the calcined catalyst hydrogel-precursor. The catalysts of the invention comprise amorphous titanium doped silica comprising pentahedrally coordinated titanium species.

The present invention relates to processes for preparing propylene oxide, comprising reaction of propene with ethylbenzene hydroperoxide in the presence of a catalyst, as well as to catalysts employed in such processes and to methods for their manufacture.

### BACKGROUND

Processes for the epoxidation of olefins to oxirane products, typically involve reaction of olefins with organic hydroperoxides in the presence of heterogeneous catalysts. A commonly known method for manufacturing propylene oxide involves the co-production of propylene oxide and styrene monomer starting from ethyl benzene, a process sometimes referred to as the propylene oxide/styrene monomer (PO/SM)-process. In general, this process involves the steps of
(i) reacting ethylbenzene (EB) with oxygen to form ethylbenzene hydroperoxide (EBHP),
(ii) reacting ethylbenzene hydroperoxide (EBHP) thus obtained, with propene in the presence of an epoxidation catalyst to yield propylene oxide (PO) and 1-phenyl ethanol (methyl phenyl carbinol, MPC), and
(iii) dehydrating 1-phenyl ethanol (MPC) into styrene (SM) using a suitable dehydration catalyst.

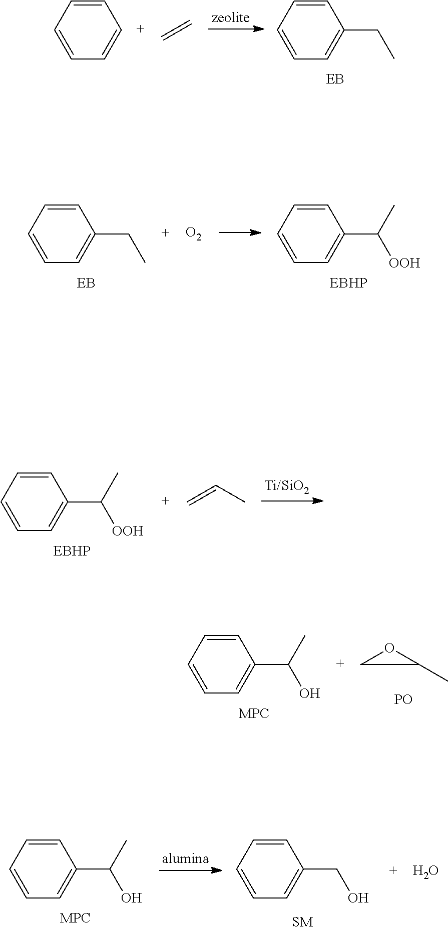

A number of titanium-containing siliceous materials have been disclosed in the prior art as catalysts for the central epoxidation step. More specifically, it has been disclosed in the prior art, that crystalline zeolitic materials comprising pentahedrally coordinated titanium species exhibit catalytic activity in epoxidation reactions (Zuo Y et al., RSC Adv., 2015, 5, 17897, Role of pentahedrally coordinated titanium in titanium silicalite-1 in propene epoxidation). Amorphous silica materials comprising pentahedrally coordinated titanium species, on the other hand, have not been suggested as catalysts for that purpose. In fact, amorphous silica materials comprising pentahedrally coordinated titanium species have not been disclosed in the prior art at all, because until now it was unknown how to make such materials.

Prior art methodology for obtaining titanium doped amorphous silica includes vapor phase deposition of titanium species on dry silica (EP1572362A1), titanium-doping of dry amorphous silica material in anhydrous, organic solvent (U.S. Pat. No. 5,932,751), titanium-doping of amorphous silica by contacting dry amorphous silica material with a solution of titanium tetrachloride, nitric acid and concentrated hydrogen peroxide (U.S. Pat. No. 4,367,342A), titanium-doping of amorphous silica by contacting dry amorphous silica material with a solution of titanium oxysulphate (US2015182959A1) and generating titanium doped silica from organic precursors of titanium and silica (US2018147560A1). However, none of these methods yields amorphous silica with pentahedrally coordinated titanium species.

### THE PRESENT INVENTION

The present invention is based on the discovery of amorphous silica materials comprising pentahedrally coordinated titanium species as well as robust processes for their manufacture, and further, the finding that such materials exhibit substantial catalytic activity for the transformation of propylene into propylene oxide.

The catalysts of the present invention based on amorphous titanium doped silica, comprising pentahedrally coordinated titanium species, exhibit high activity and selectivity towards target product in processes for transforming propylene into propylene oxide in the propylene oxide/styrene monomer (PO/SM)-process and the catalyst-manufacturing processes of the present invention can be adapted to large scale industrial production.

The processes of the present invention, directed at manufacturing catalysts comprising amorphous titanium doped silica, comprising pentahedrally coordinated titanium species, comprise the following steps:
a. sol-gel-synthesis of catalyst hydrogel-precursor, comprising the following steps:
   i. adding water glass exhibiting a viscosity in the range of 400 to 600 mPa*s into an acid, thereby yielding silica gel exhibiting residual water content Xwa in the range between Xw1 and Xw2,
   ii. removing impurities from said silica gel by washing the silica gel with an acidic aqueous solution, thereby yielding purified silica gel,
   iii. forming catalyst hydrogel-precursor, alternative a:
      doping said purified silica gel by contacting the purified silica gel with an acidic aqueous solution of a titanium salt, thereby yielding titanium-doped silica gel, and
      aging said titanium-doped silica gel by suspending the titanium-doped silica gel in an aging-solution for a time span in the range between 4 h and 6 h at a temperature in the range between 60° C. and 90° C., wherein the liquid part of the suspension formed thereby exhibits a pH value in the range of 5.5 to 7.5, thereby yielding catalyst hydrogel-precursor exhibiting titanium content Xt in the range between Xt1 and Xt2,
      or
      forming catalyst hydrogel-precursor, alternative b:
      aging said purified silica gel by suspending the purified silica gel in an aging-solution for a time span in the range between 4 h and 6 h at a temperature in the range between 60° C. and 90° C., wherein the liquid part of the suspension formed thereby exhibits a pH value in the range of 5.5 to 7.5, thereby yielding aged, purified silica gel, and
      doping said aged, purified silica gel by contacting the aged, purified silica gel with an acidic aqueous solution of a titanium salt, thereby yielding catalyst hydrogel-precursor exhibiting titanium content Xt in the range between Xt1 and Xt2,
b. drying of catalyst hydrogel precursor to a residual water content Xwb of the dried catalyst hydrogel-precursor in a range between Xw3 and Xw4,
c. calcining of dried catalyst hydrogel-precursor,
   wherein
   Xw1=40 wt %, Xw2=60 wt %,
   Xt1=0.85 wt %, Xt2=1.9 wt %,
   Xw3=1 wt %, Xw4=7 wt %,
   and wherein
   residual water content Xwa and Xwb of samples is determined as the weight loss exhibited by a 100 g-aliquot after drying at 200° C. for four hours in a drying cabinet under atmospheric conditions,
   and wherein
   titanium content Xt of samples is determined by elemental analysis via optical emission spectrometry with inductively coupled plasma (ICP-OES) after dissolving samples by adding 5 ml of ultrapure water, 1.5 ml of 0.2 wt % aqueous D-mannitol-solution and 5 ml of 40 wt %-50 wt % aqueous HF solution to 1 g of sample, followed by removing liquid components by heating to 120° C., and, successively, dissolving the remaining residue in 1 ml 65 wt %-70 wt % aqueous HNO₃ solution and 2 ml H₂O₂ under heat treatment at 120° C. for 1 h, and diluting the resulting liquid tenfold with ultrapure water prior to measurement,
   and wherein
   Ti-contents of solid materials are calculated on a dry-mass basis and correspond to the content of elemental titanium.

Processes for the manufacture of catalysts of the present invention start with the sol-gel-synthesis of catalyst hydrogel-precursor. It is important in this context to appreciate differences between various types of silica-gel. In the context of the present application, the following definitions will be adhered to:
(1) A Silica Hydrogel is a material made via a sol-gel process by mixing sulfuric acid and sodium (or potassium) water glass carried out in an aqueous system. The water content of such a hydrogel gel depends on the ratio of sulfuric acid to water glass and is typically in the range between 20 and 80 wt %.
(2) A Silica Xerogel is a material made by drying a silica hydrogel according to definition (1) to a water content below 20 wt %.
(3) A Metal-Silica-Cogel Hydrogel type I is a material made via a sol-gel process by mixing sulfuric acid, a metal salt solution and sodium (or potassium) water glass carried out in an aqueous system. The water content of such a Metal-Silica-Cogel hydrogel type I depends on the sulfuric acid, the metal salt to water glass ratio and is typically in the range between 20 and 80 wt %. Typically, a metal sulfate is being used as a metal salt.
(4) A Metal-Silica-Cogel Xerogel type I is a material made by drying a Metal-Silica-Cogel hydrogel type I according to definition (3) to a water content below 20 wt %.
(5) A Metal-Silica-Cogel Hydrogel type II is a material made via a sol-gel process to get first a silica hydrogel according to definition (1). After the gelation of the silica hydrogel an aqueous metal salt solution is used to impregnate the silica hydrogel (prior to a drying step). Wherein impregnation means adding a liquid solution of a metal salt to a non-liquid substance in order to deposit the metal onto that substance. The water content of such a Metal-Silica-Cogel hydrogel type II is typically in the range between 20 and 80 wt %.
(6) A Metal-Silica-Cogel Xerogel type II is a material made by drying a Metal-Silica-Cogel hydrogel type II according to definition (5) to a water content below 20 wt %.
(7) A Metal-Silica-Cogel Xerogel type III is a material made via a sol-gel process according to definition (2). After the preparation of a Silica Xerogel a metal salt solution is used to impregnate the silica xerogel (after the drying step). Wherein impregnation means adding a liquid solution of a metal salt to a non-liquid substance in order to deposit the metal onto that substance. Normally the impregnated material is dried after impregnation in order to remove the remaining impregnation solution. The impregnation solution can be water-based or non-water-based. The water content of such a Metal-Silica-Cogel Xerogel type III is below 20 wt %.

The catalysts of the present invention are based on Metal-Silica-Cogel hydrogels of type II according to definition (5) obtained after step (a) (iii) of the process with Titanium as metal dopant (aqueous Titanylsulfate solution is used to impregnate the silica hydrogel) and Metal-Silica-Cogel Xerogels of type II according to definition (6) obtained after step (b) of the process.

In a first step of the processes for the manufacture of catalysts of the present invention water glass is added into an acid in a fritted flow reactor to obtain silica gel. Corresponding processes for obtaining silica gel are well known in the art (cf. e.g., The colloid chemistry of silica and silicates, Ralph K. Iler, Cornell University Press, Ithaca, New York, 1955). A suitable acid for this process is dilute sulfuric acid at a concentration of 5 to 50 wt %, however other acids may be selected by persons of skill in the art. The water glass employed in this step is required to exhibit a viscosity in the range of 400 to 600 mPa*s. After contacting the acid, the drops of water glass instantly sink to the bottom of the reactor forming a bed of silica gel particles. In order to obtain material comprising titanium doped amorphous silica, comprising pentahedrally coordinated titanium species according to the invention, the residual water content Xwa of these silica gel particles needs to be in the range between 40 and 60 wt %.

It is important to note in this context, that the water content of the silica gel particles obtained at this stage of the process (i.e. at the end of step (a) (i)) undergoes no significant change until the end of step (a) (iii) of the process. The water content of the silica gel particles obtained at this stage of the process (i.e. at the end of step (a) (i)) is, thus, substantially the same as the water content of the catalyst hydrogel-precursor material obtained at the end of step (a) (iii). Accordingly, it does not matter if residual water content is determined at the end of process step (a) (i) or at the end of step (a) (iii) in order to determine if Xwa is in the range between Xw1 and Xw2.

The viscosity of the water glass employed in this step is the key determinant of the residual water content of the silica gel particles obtained. Increasing viscosity of the water glass used at this stage will decrease residual water content of the silica gel particles obtained and vice versa. In accord with this, it is required to use water glass exhibiting a viscosity in the range of 400 to 600 mPa*s in this step.

Residual water content of materials obtained, is determined as the weight loss exhibited by a 100 g-aliquot after drying at 200° C. for four hours in a drying cabinet under atmospheric conditions.

In the following step, impurities are removed from the silica gel by circulating an acidic aqueous solution through the bed of gel particles. Typically, this process is performed for about 2 h, while recycling the acidic aqueous solution by continuously pumping it through an ion exchange column (typically styrene-divinylbenzene functionalized with aminophosphonic acid) in order to remove metal cations. This washing step can be repeated at elevated temperatures (typically about 50 to 70° C.) with fresh acidic aqueous solution in order to remove additional impurities until the following stoichiometric purity-level is obtained: Total metal content<1%, Sb≤0.1 ppm, Sn≤0.1 ppm, Zn≤0.0 ppm, Zr≤0.2 ppm, Al≤0.8 ppm, As<0.01 ppm, B<0.01 ppm, Ba≤2.2 ppm, Ca≤0.3 ppm, Cr≤0.1 ppm, Cu≤0.0 ppm, Fe≤2.9 ppm, K≤0.1 ppm, Mg≤0.0 ppm, Na≤1.5 ppm, Ni<0.01 ppm. Finally, the acid is discharged from the reactor and the gel particles are rinsed with deionized water, typically for about 1 h.

In the next step the catalyst hydrogel-precursor is formed from the purified silica gel obtained in the previous step. Two alternative procedures can be pursued in order to achieve this:
In alternative a the purified silica gel is first doped by contacting the purified silica gel with an acidic aqueous solution of a titanium salt, thereby yielding titanium-doped silica gel, followed by aging the titanium-doped silica gel by suspending the titanium-doped silica gel in an aging-solution for a time span in the range between 4 h and 6 h at a temperature in the range between 60° C. and 90° C., wherein the liquid part of the suspension formed thereby exhibits a pH value in the range of 5.5 to 7.5, thereby yielding catalyst hydrogel-precursor exhibiting titanium content Xt in the range between Xt1 and Xt2.
In alternative b the purified silica gel is aged by suspending the purified silica gel in an aging-solution for a time span in the range between 4 h and 6 h at a temperature in the range between 60° C. and 90° C., wherein the liquid part of the suspension formed thereby exhibits a pH value in the range of 5.5 to 7.5, thereby yielding aged purified silica gel, followed by doping the aged purified silica gel by contacting the aged purified silica gel with an acidic aqueous solution of a titanium salt, thereby yielding catalyst hydrogel-precursor exhibiting a titanium content Xt in the range between Xt1 and Xt2,

For the purpose of doping with titanium, a dopant solution of a titanium salt in dilute acid (typically titanyl-sulfate (TiO(SO₄)) at a concentration in the range between 2 and 10 wt % in sulfuric acid at a concentration of about 20 to 25 wt %) is contacted with the purified silica gel or with the aged purified silica gel in the reactor, typically for about 1 h. The titanium concentration in the doped product can be increased by increasing the titanium concentration in the dopant solution, by increasing the amount of dopant solution contacted with the silica gel and by increasing the duration of contact between dopant solution and silica gel. Likewise, decreasing the respective physical quantities, decreases the titanium concentration in the doped product. Afterwards the titanium-doped silica gel is washed with deionized water.

For the purpose of aging the purified silica gel or the titanium-doped silica gel is suspended in an aging-solution for a time span in the range between 4 h and 6 h at a temperature in the range between 60° C. and 90° C., followed by washing with deionized water. The aging solution needs to impart to the liquid part of the suspension formed, a pH value in the range of 5.5 to 7.5. Further, the aging-solution needs to consist, exclusively, of components that can be removed entirely by heating to about 300° C. Dilute ammonium acetate solution (1 to 50 wt %) exhibiting a pH in the range between 5.5 and 6.5 is a typical example of such an aging-solution. It is important to adhere to the pH range of 5.5 to 7.5 for the liquid part of the suspension, because aging affects significant textural properties of the silica matrix, i.e. specific BET surface area A_{S,BET}, average pore-width D_{P,BJH} and pore-volume V_{P,total}, as documented in the academic literature (e.g., The influence of pH on the surface characteristics of silica gel soaked in aqueous solutions, D. Dollimore, G. M. Heal, Journal of Applied Chemistry, 1962, 12, 445-450; The colloid chemistry of silica and silicates, Ralph K. Iler, Cornell University Press, Ithaca, New York, 1955). A pH value below the desired range will lead to a higher specific BET surface area A_{S,BET}, lower average pore-width D_{P,BJH} and slightly lower pore-volume V_{P,total} while a pH value above the desired range will give materials with lower specific BET surface area A_{S,BET}, higher average pore-width D_{P,BJH} and slightly higher pore-volume V_{P,total}. At a constant pH value (in the desired range) an increase in temperature leads to a decrease in specific BET surface area A_{S,BET}, increase in average pore-width D_{P,BJH} and slightly increase in pore-volume V_{P,total} while with a decrease in temperature, an increase in specific BET surface area A_{S,BET}, decrease in average pore-width D_{P,BJH} and slight decrease in pore-volume V_{P,total} may be observed. The textural properties are chosen in order to fit the size of molecules to be converted selectively into desired products in the catalytic reaction.

At the end, the water is rinsed off and catalyst hydrogel-precursor is obtained exhibiting a residual water content between 40 wt % and 60 wt % and a titanium content between 0.85 and 1.9 wt %. The titanium content of the samples is determined by elemental analysis via optical emission spectrometry with inductively coupled plasma (ICP-OES) after dissolving as follows: to 1 g of sample 5 ml of ultrapure water, 1.5 ml of D-mannitol-solution (1 g D-mannitol in 499 g ultrapure water) and 5 ml HF (40-50 wt % aqueous solution) are added. The resulting solution is then heated to 120° C. to remove the liquid. The remaining residue is successively dissolved in 1 ml HNO₃ (65-70 wt % aqueous solution) und 2 ml H₂O₂ under heat treatment at 120° C. for 1 h. The resulting liquid is diluted 1/10 with ultrapure water prior to the measurement.

Ti-contents of solid materials referred to in the present invention are/have been calculated on a dry-mass basis. Residual water content of materials obtained, is determined as the weight loss exhibited by a 100 g-aliquot after drying at 200° C. for four hours in a drying cabinet under atmospheric conditions.

The catalyst hydrogel precursor, subsequently, is dried (typically at a temperature between 15° and 170° C. with heating rates of about 2 to 5° C./min) to a residual water content in the range between 1 and 7 wt %, yielding dried catalyst hydrogel-precursor. Drying can be performed, e.g., in a drying cabinet under atmospheric conditions. Residual water is determined as the weight loss exhibited by a 100 g-aliquot after drying at 200° C. for four hours in a drying cabinet under atmospheric conditions.

The dried catalyst hydrogel-precursor is calcined afterwards (typically at temperatures of around 800° C.), yielding calcined catalyst hydrogel-precursor. Calcining can be performed in a furnace under atmospheric conditions.

In a preferred embodiment of the present invention, the calcined catalyst hydrogel-precursor is, furthermore, hydrophobized with a hydrophobizing-agent (which is a tri- or tetra-substituted organosilane such as, e.g., dimethyltrimethylsilylamine, bis(dimethylamino) diethylsilane, hexamethyldisilazane) in liquid phase. For this purpose, a hydrophobizing agent is dissolved in a solvent (typically a high-boiling solvent such as an aromatic high-boiling solvent) and contacted with the calcined catalyst hydrogel-precursor under stirring. Afterwards, the solid material is filtered off and dried, yielding the catalyst.

Titanium contents of all solid materials referred to in the context of the present invention were determined by elemental analysis via optical emission spectrometry with inductively coupled plasma (ICP-OES). Prior to measurement samples were completely dissolved as follows: to 1 g of sample 5 ml of ultrapure water, 1.5 ml of D-mannitol-solution (1 g D-mannitol in 499 g ultrapure water) and 5 ml HF (40-50 wt % aqueous solution) are added. The resulting solution is then heated to 120° C. to remove the liquid. The remaining residue is successively dissolved in 1 ml HNO₃ (65-70 wt % aqueous solution) und 2 ml H₂O₂ under heat treatment at 120° C. for 1 h. The resulting liquid is diluted 1/10 with ultrapure water prior to the measurement.

Titanium contents of all solid materials referred to in the context of the present invention are/have been calculated on a dry-mass basis. Titanium contents of all solid materials referred to, correspond to the content of elemental titanium. The difference between titanium contents obtained accordingly, for samples of catalyst hydrogel precursor, i.e. material obtained after step (a) (iii) of the process of the invention, and titanium contents obtained, for samples of the same material batch that had, additionally, undergone any or all of the subsequent steps of the process of the present invention (i.e. steps b, c, d), was negligible, i.e. less than one hundredth of the values obtained.

Catalysts were characterized with respect to their specific BET surface area A_{S,BET}, average pore-width D_{P,BJH}, and pore-volume V_{P,total}. Specific BET surface area A_{S,BET}, average pore-width D_{P,BJH} and pore-volume V_{P,total} can be derived from nitrogen physisorption isotherms as measured, e.g., on a Micromeritics ASAP 2420 analyzer at 77 K. Prior to measurement, individual samples have to be outgassed in vacuum at 40-50 mbar for 10 h at 300° C. for calcined materials and 10 h at 110° C. for hydrophobized materials. The Brunnauer-Emmett-Teller (BET) method according to DIN 9277:2014 was utilized to calculate the specific surface areas A_{S,BET}. The average pore width D_{P,BJH} as well as total pore-volume V_{P,total} was calculated by applying the Barrett, Joyner and Halenda (BJH) method using the desorption branches of the isotherms (cf. Journal of the American Chemical Society, 73:373-380, 1951).

Pentahedrally coordinated titanium species can be detected in siliceous matrices by their specific spectroscopic signature, i.e.
a. a DR-UV/Vis band maximum between 220 and 245 nm,
b. a DR-UV/Vis derived share of intensity below 275 nm (SIb275) of at least 75%, wherein SIb275 is determined by measuring Diffuse Reflectance (DR)-UV/Vis of the material, with the measured reflection calculated to Kubelka-Munk-units, and dividing the integrated intensity measured between 200 and 275 nm (Ai, 275 nm) by the integrated intensity measured between 200 and 375 nm (Ai, total) according to: SIb275=(Ai, 275 nm)/(Ai, total).

Accordingly, the materials obtained were characterized by Diffuse Reflectance (DR)-UV/Vis with the measured reflection calculated to Kubelka-Munk-units (cf. Reflectance Spectroscopy, W. WM. Wendlandt, H. G. Hecht, Interscience Publishers/John Wiley NY, 1966; Reflectance Spectroscopy G. Kortüm, Springer Berlin, 1969; Interpreting Diffuse Reflectance and Transmittance: A Theoretical Introduction to Absorption Spectroscopy of Scattering Materials, D. J. Dahm, K. D. Dahm, IM Publications Open LLP Chichester, 2007) with respect to their share of intensity below 275 nm (defined herein as SI_{b275}) and position of band maximum (highest point of curve). The share of intensity below 275 nm is calculated by dividing the integrated intensity between 200 and 275 nm (A_{i, 275 nm}) by the integrated intensity of the entire band between 200 and 375 nm (A_{i, total}) according to Eq. 1. Band integration in order to obtain the integrated intensity is done as for chromatographic peak integration (cf. Gas Chromatography, I. A. Folis, Wiley & Sons Ltd, 1995).

$$share of intensity below 275 nm [ \% ] = ( \frac{{A}_{i , 275 nm}}{{A}_{i , total}} ) * 100 \qquad Eq \text{.1}$$

In one aspect the present invention relates to catalysts for transforming propylene into propylene oxide in the propylene oxide/styrene monomer-process, comprising titanium doped amorphous silica, comprising pentahedrally coordinated titanium species.

Furthermore, the present invention relates to catalysts obtainable by the catalyst manufacturing processes of the present invention.

In the course of investigations underlying the present invention it was found, that particularly beneficial catalytic properties can be obtained for catalysts exhibiting titanium content within particular ranges. Accordingly, in a preferred embodiment of the present invention Xt is selected in the range between Xt1=1 wt % and Xt2=1.9 wt %. In a particularly preferred embodiment of the present invention Xt is selected in the range between Xt1=1.4 wt % and Xt2=1.8 wt %. In another particularly preferred embodiment of the present invention Xt is selected in the range between Xt1=1.6 wt % and Xt2=1.8 wt %.

Furthermore, in the context of the present invention it was found that catalysts with particularly beneficial catalytic properties can be obtained when the catalyst hydrogel-precursor exhibits residual water content Xwa in a particular range. Accordingly, in a preferred embodiment of the present invention the catalyst hydrogel-precursor exhibits residual water content Xwa in the range between Xw1=45 wt % and Xw2=60 wt %. %. In another preferred embodiment of the present invention Xwa of the catalyst hydrogel-precursor is selected in the range between Xw1=47 wt % and Xw2=60 wt %. In a particularly preferred embodiment of the present invention Xwa of the catalyst hydrogel-precursor is selected in the range between Xw1=52 wt % and Xw2=57 wt %.

A preferred embodiment of the present invention, relates to catalysts for transforming propylene into propylene oxide in the propylene oxide/styrene monomer-process, exhibiting a DR-UV/Vis derived share of intensity below 275 nm (SI_{b275}) of at least 75%, and a DR-UV/Vis band maximum between 220 and 245 nm. In a more preferred embodiment, the present invention relates to catalysts for transforming propylene into propylene oxide in the propylene oxide/styrene monomer-process exhibiting a DR-UV/Vis derived share of intensity below 275 nm (SI_{b275}) of at least 90% and a DR-UV/Vis band maximum between 220 and 245 nm.

In the context of the present invention it was examined if structural parameters like specific BET surface area A_{S,BET}, average pore-width D_{P,BJH} and pore-volume V_{P,total}, of the catalyst according to the present invention, could be tailored to the requirements for transforming propylene into propylene oxide in the propylene oxide/styrene monomer-process. It was found that, indeed, selecting these parameters in certain ranges, yields particularly effective catalysts.

Accordingly, in a preferred embodiment the present invention relates to catalysts for transforming propylene into propylene oxide in the propylene oxide/styrene monomer-process, exhibiting specific BET surface area As, BET in the range between 350 and 650 m²/g. In a more preferred embodiment, the present invention relates to catalysts for transforming propylene into propylene oxide in the propylene oxide/styrene monomer-process, exhibiting specific BET surface area As. BET in the range between 400 and 550 m²/g.

Further, in a preferred embodiment the present invention relates to catalysts for transforming propylene into propylene oxide in the propylene oxide/styrene monomer-process, exhibiting average pore-width D_{P, BJH} in the range between 4 and 8 nm, in a more preferred embodiment, the present invention relates to catalysts for transforming propylene into propylene oxide in the propylene oxide/styrene monomer-process, exhibiting average pore-width D_{P, BJH} in the range between 4 and 5 nm,
wherein average pore-width D_{P,BJH} is derived from nitrogen physisorption isotherms as measured on a Micromeritics ASAP 2420 analyzer at 77 K, where prior to measurement, individual samples are outgassed in vacuum at 40-50 mbar for 10 h at 300° C. for calcined materials and for 10 h at 110° C. for hydrophobized materials and average pore width D_{P,BJH} is calculated by applying the Barrett, Joyner and Halenda (BJH) method using the desorption branches of the isotherms as described in Journal of the American Chemical Society, 73:373-380, 1951.

Further, in a preferred embodiment the present invention relates to catalysts for transforming propylene into propylene oxide in the propylene oxide/styrene monomer-process, exhibiting pore-volume V_{P, total} in the range between 0.4 and 1.0 cm³/g, in a more preferred embodiment, the present invention relates to catalysts for transforming propylene into propylene oxide in the propylene oxide/styrene monomer-process, exhibiting pore-volume V_{P, total} in the range between 0.7 to 0.9 cm³/g,
wherein pore-volume V_{P,total} is derived from nitrogen physisorption isotherms as measured on a Micromeritics ASAP 2420 analyzer at 77 K, where prior to measurement, individual samples are outgassed in vacuum at 40-50 mbar for 10 h at 300° C. for calcined materials and for 10 h at 110° C. for hydrophobized materials and pore-volume V_{P,total} is calculated by applying the Barrett, Joyner and Halenda (BJH) method using the desorption branches of the isotherms as described in Journal of the American Chemical Society, 73:373-380, 1951.

In a particularly preferred embodiment, the present invention relates to catalysts for transforming propylene into propylene oxide in the propylene oxide/styrene monomer-process exhibiting the following:
a. titanium content in the range between 1.6 and 1.8 wt %,
b. specific BET surface area As, BET between 400 and 450 m²/g,
c. average pore-width D_{P, BJH} between 4.5 and 5 nm,
d. pore-volume V_{P, total} between 0.7 and 0.8 cm³/g
   titanium content Xt of samples is determined by elemental analysis via optical emission spectrometry with inductively coupled plasma (ICP-OES) after dissolving samples by adding 5 ml of ultrapure water, 1.5 ml of 0.2 wt % aqueous D-mannitol-solution and 5 ml of 40 wt %-50 wt % aqueous HF solution to 1 g of sample, followed by removing liquid components by heating to 120° C., and, successively, dissolving the remaining residue in 1 ml 65 wt %-70 wt % aqueous HNO₃ solution and 2 ml H₂O₂ under heat treatment at 120° C. for 1 h, and diluting the resulting liquid tenfold with ultrapure water prior to measurement,
   and wherein
   Ti-contents of solid materials are calculated on a dry-mass basis and correspond to the content of elemental titanium,
   wherein average pore-width D_{P, BJH} is derived from nitrogen physisorption isotherms as measured on a Micromeritics ASAP 2420 analyzer at 77 K, where prior to measurement, individual samples are outgassed in vacuum at 40-50 mbar for 10 h at 300° C. for calcined materials and for 10 h at 110° C. for hydrophobized materials and average pore width D_{P, BJH} is calculated by applying the Barrett, Joyner and Halenda (BJH) method using the desorption branches of the isotherms as described in Journal of the American Chemical Society, 73:373-380, 1951,
   wherein pore-volume V_{P,total} is derived from nitrogen physisorption isotherms as measured on a Micromeritics ASAP 2420 analyzer at 77 K, where prior to measurement, individual samples are outgassed in vacuum for 10 h at 300° C. for calcined materials and for 10 h at 110° C. for hydrophobized materials and pore-volume V_{P,total} is calculated by applying the Barrett, Joyner and Halenda (BJH) method using the desorption branches of the isotherms as described in Journal of the American Chemical Society, 73:373-380, 1951.

Another aspect of the present invention relates to oxidation reactions performed in the presence of catalysts according to the invention. More specifically, the present invention relates to processes for the preparation of propylene oxide comprising the reaction of propene with ethylbenzene hydroperoxide in the presence of catalysts according to the present invention. In particular, the present invention relates to processes for the preparation of propylene oxide comprising the reaction of propene with ethylbenzene hydroperoxide in the presence of catalysts obtainable by the catalyst manufacture processes of the present invention.

The catalysts of the present invention are used in oxidation reactions, being particularly useful for catalyzing the epoxidation of olefins with an organic hydroperoxide. Olefin epoxidation processes are well known in the art.

In a particularly preferred embodiment, the catalyst obtained in accordance with the method of the present invention is used in the co-production of propylene oxide and styrene starting from ethyl benzene. In general, such processes involve the steps of reacting ethyl benzene (EB) with oxygen or air to form ethyl benzene hydroperoxide (EBHP); reacting the ethylbenzene hydroperoxide (EBHP) thus obtained with propene in the presence of an epoxidation catalyst to yield propylene oxide (PO) and 1-phenyl ethanol (methyl phenyl carbinol, MPC); and dehydrating the 1-phenyl ethanol (MPC) into styrene (SM) using a suitable dehydration catalyst.

In the epoxidation reaction, the molar ratio of olefin to hydroperoxide can vary over a wide range and a molar excess of either the olefin or hydroperoxide of up to as high as 100:1 can be used. Preferably, molar ratios of olefin to hydroperoxide varying from about 50:1 to about 1:10 are satisfactory, although it is particularly preferred to employ molar ratios of olefin to hydroperoxide of about 20:1 to about 1:1.

The organic hydroperoxide is prepared by direct oxidation methods, such methods being well-known in the art. For example, molecular oxygen may be passed through the hydrocarbon to convert a portion of the hydrocarbon to the corresponding organic hydroperoxide. Either pure oxygen, air, or oxygen combined with an inert gas such as nitrogen can be used.

In addition to the propylene oxide formation reaction, a secondary reaction occurs in the epoxidation stage by the catalytic decomposition of EBHP, in which acetophenone (ACP) is generated as a side-product. This side reaction leads to a lower selectivity of the hydroperoxide to propylene oxide. Typically, the selectivity of EBHP to ACP is in the range of 4 to 8%.

The epoxidation reaction may be conducted in the liquid-phase in solvents or diluents that are liquid at the reaction temperature and pressure and are substantially inert to the reactants and the products produced therefrom. Generally, the same hydrocarbon used to produce the organic hydroperoxide reactant may be used as a solvent. For example, when ethyl benzene hydroperoxide is utilized, the use of ethyl benzene as the epoxidation solvent is preferred.

Optionally, the ethylbenzene hydroperoxide is pretreated prior to epoxidation reaction. This pretreatment includes at least a basic washing and distillation steps. A typical pretreatment for ethylbenzene hydroperoxide is described for instance in U.S. Pat. No. 5,883,268A.

The epoxidation reaction may be conducted in the liquid-phase in solvents or diluents that are liquid at the reaction temperature and pressure and are substantially inert to the reactants and the products produced therefrom. Generally, the same hydrocarbon used to produce the organic hydroperoxide reactant may be used as a solvent. For example, when ethyl benzene hydroperoxide is utilized, the use of ethyl benzene as the epoxidation solvent is preferred.

Suitable reaction temperatures vary from room temperature to 200° C., preferably from room temperature to 160° C., more preferably from 70° C. to 120° C. The reaction is preferably conducted at or above atmospheric pressure. Typical pressures vary from 1 atmosphere to 100 atmospheres, preferably from 40 atmosphere to 90 atmospheres.

The epoxidation may be performed using any of the conventional reactor configurations known in the art for reacting olefin and organic hydroperoxides in the presence of an insoluble catalyst. Continuous as well as batch procedures may be used.

One preferred disposal for continuous fixed bed processes is a series of reactors loaded with catalyst. The EBHP solution can be split into different reactors. A preferred operation method for this kind of epoxidation facility making use of heterogeneous catalysts is described for instance in EP1047681A1.

In the context of the present invention, the terms “directly contacting” or “directly drying” or synonyms, denote that no intermediate step is performed between the previous step and the referenced step (contacting, drying, . . . ).

Throughout the description and claims the word “comprise” and grammatic variations of the word, are not intended to exclude other technical features, additives, components, or steps. Additional objects, advantages and features of the invention will become apparent to those skilled in the art upon examination of the description or may be learned by practice of the invention. The following examples are provided by way of illustration, and they are not intended to be limiting of the present invention.

### EXAMPLES

### Example 1

### Catalyst 1

1800 liter of diluted sulfuric acid (16 wt %) were filled in a fixed-bed reactor. Then 630 l of water glass (viscosity of 500 mPa*s) were prilled into the diluted sulfuric acid. The water glass drops gelled instantly after contact with the acid and formed gel particles. The gel particles sank down and formed a bed of gel particles in the reactor.

The sulfuric acid was circulated over the bed of gel particles in the reactor and an ion exchange column with a flow rate of 1600-2000 l per h. The acid was intended to remove three, four and five valence metal ion impurities from the gel particles and transport them onto the ion exchange column where they are adsorbed. The cycling was continued for approximately 75-80 min (depending on the desired purity).

Then the acid was replaced with fresh diluted sulfuric acid. The acid was heated to 60° C. During heating the acid was again cycled over the bed and the ion exchange column. Cycling was performed at a flow rate of 4000 l per hour for 80 min.

Then the acid was discharged, and the gel particles were rinsed with deionized water for 60 min with a flow rate of 8000 l per h.

A solution of sulfuric acid (25 wt %) and titanium sulfate (3-4 wt %, aiming at a titanium content of the catalyst hydrogel precursor of 1.7 wt %) was cycled over the fixed bed gel particles in the reactor for 120 min.

The solution was replaced with deionized water and the gel particles were washed with deionized water for with a flow rate of 10000 l per h. 80 l of a solution of ammonium acetate (50 wt %) were dosed into the stream of deionized water, thereby ammonium acetate concentration was adjusted to approx. 7.5 wt % and the pH value to was raised to 5.5-6.5. This solution was cycled over the fixed bed gel particles in the reactor for 5-6 h at 80-85° C.

Afterwards, the ammonium acetate solution was replaced by deionized water and washed with deionized water for 60 minutes with a flow rate of 8000 l per h.

At the end, the water was rinsed off and the titanium doped silica hydrogel was discharged from the reactor. The as synthesized hydrogel possesses a residual water content, Xwa, of approximately Xwa=55.5 wt % and a Ti-content of 1.69 wt % (2.82 wt % TiO₂). The titanium content of the samples was determined by elemental analysis via optical emission spectrometry with inductively coupled plasma (ICP-OES) after dissolving the solids as follows: to 1 g of sample 5 ml of ultrapure water, 1.5 ml of D-mannitol-solution (1 g D-mannitol in 499 g ultrapure water) and 5 ml HF (40-50 wt % aqueous solution) are added. The resulting solution is then heated to 120° C. to remove the liquid. The remaining residue is successively dissolved in 1 ml HNO₃ (65-70 wt % aqueous solution) und 2 ml H₂O₂ under heat treatment at 120° C. for 1 h. The resulting liquid is diluted 1/10 with ultrapure water prior to the measurement.

Ti-contents of solid materials referred to in the present invention have been calculated on a dry-mass basis.

500 g of the hydrogel that had been obtained was then transferred to a drying cabinet, heated up to 160° C. applying a heating rate of 3° C./min and kept at this temperature for 15 h in air atmosphere. For the resulting material a residual water content, Xwb, of Xwb=3.2 wt % was found.

Thereafter, the dried material was calcined in a furnace under static conditions for 2 h at 800° C. (applying a heating rate of 5° C./min to reach the desired temperature), resulting in a material characterized by a specific BET surface area A_{S,BET} of 491 m²/g, an average pore-width D_{P,BJH} of 5.3 nm and a pore-volume V_{P,total} of 0.8 cm³/g. The specific BET surface area A_{S,BET}, average pore-width D_{P,BJH} and pore-volume V_{P,total} derived from nitrogen physisorption isotherms, measured on a Micromeritics ASAP 2420 analyzer at 77 K. Prior to the measurements, the individual samples were outgassed in vacuum at 40-50 mbar for 10 h at 300° C. for calcined materials and 10 h at 110° C. for hydrophobized materials. The Brunnauer-Emmett-Teller (BET) method according to DIN 9277:2014 was utilized to calculate the specific surface areas A_{S,BET}. The average pore width D_{P,BJH} as well as total pore-volume V_{P,total} was calculated by applying the Barrett, Joyner and Halenda (BJH) method using the desorption branches of the isotherms (cf. Journal of the American Chemical Society, 73:373-380, 1951).

As indicated by Diffuse Reflectance (DR)-UV/Vis with the measured reflection calculated to Kubelka-Munk-units (cf. Reflectance Spectroscopy, W. WM. Wendlandt, H. G. Hecht, Interscience Publishers/John Wiley NY, 1966; Reflectance Spectroscopy G. Kortüm, Springer Berlin, 1969; Interpreting Diffuse Reflectance and Transmittance: A Theoretical Introduction to Absorption Spectroscopy of Scattering Materials, D. J. Dahm, K. D. Dahm, IM Publications Open LLP Chichester, 2007), the dried material further showed a share of intensity below 275 nm of 90% and a band maximum centered at 233±5 nm. The respective DR-UV/Vis spectra were collected on a Varian Cary5000 spectrophotometer, equipped with a diffuse reflectance (DR) sphere using spectralon® (PTFE, reflective value 99%) as a reference. Samples were activated in a desiccator under vacuum for at least 5 h prior to each measurement. Materials were characterized by share of intensity below 275 nm SI_{b275} and position of band maximum (highest point of curve). The share of intensity below 275 nm is calculated by dividing the integrated area between 200 and 275 nm (A_{i, 275} nm) by the integrated area of the entire band between 200 and 375 nm (A_{i, total}) according to Eq. 1. Band integration to obtain the area is done as for chromatographic peak integration (cf. Gas Chromatography, I. A. Folis, Wiley & Sons Ltd, 1995).

$$share of intensity below 275 nm [ \% ] = ( \frac{{A}_{i , 275 nm}}{{A}_{i , total}} ) * 100 \qquad Eq \text{.1}$$

Calcination was followed by a hydrophobization step. For this purpose, 40 g of the calcined material were placed in a 3-necked round bottom flask followed by the addition of 264 ml toluene and 20 g of hexadimethylsilazane. The reaction mixture was smoothly mixed (100 min 1) for 1 hour at 25° C., subsequently heated to 110° C. and kept at this temperature for 3 hours. After cooling down to 25° C. the solid was filtered off, partially dried in a flow of nitrogen and placed into a vacuum dryer at a temperature not exceeding 110° C. overnight in order to remove the solvent.

Results of the physico-chemical characterization of catalysts are summarized in table 1.

### Evaluation of Catalyst Performance:

In order to evaluate performance of the catalyst, a continuous fixed bed epoxidation of propene with ethylbenzene hydroperoxide was carried out. Performance was tested in an isothermal fixed bed reactor (diameter=10 mm, length=1.75 m) and the temperature was controlled by a jacket with thermal oil. The catalyst loading is indicated in Table 2, depending on the test. The catalyst was mixed with glass balls (1 mm) to improve temperature control and both top and bottom had glass balls (3 mm) with a bigger diameter to protect the catalytic bed. The mixture feed was fed to the reactor from top to bottom

Epoxidation of propene was carried out with a solution of ethylbenzene hydroperoxide. A solution of 35 wt % ethylbenzene hydroperoxide in ethylbenzene, made by oxidation of ethylbenzene with air, was used. The solution was pretreated according to the procedure described in U.S. Pat. No. 5,883,268. After pretreatment, the ethylbenzene hydroperoxide solution had an acids content below 500 ppm, a water content below 5000 ppm, and it contained also some methyl phenyl ketone and/or 1-phenyl-ethanol formed in the preceding oxidation. A typical ethylbenzene hydroperoxide feed comprised 33-36 wt % ethylbenzene hydroperoxide, 53-54 wt % ethylbenzene, 2-4 wt % 1-phenyl-ethanol and 3-5 wt % methyl phenyl ketone. The propene was added to the ethylbenzene hydroperoxide feed, and the mixture was fed into the fixed bed reactor.

Ethylbenzene hydroperoxide (EBHP) solution and propene were fed in continuous in the fixed bed reactor at 25° C. Amounts of EBHP and propene were adjusted with amount of catalyst keeping the ratio between EBHP, propene and catalyst constant. For exact amounts see Table 2.

The reactor temperature was controlled at 100° C., and the reaction was carried out at a pressure of 60 bar. Once the reactor was in a continuous operation at the set temperature, samples were taken every 24 h to analyze the outlet composition by GC and HPLC, using Agilent 6850 and Agilent 1100 Series chromatographs.

In Table 2, the following results are summarized for the epoxidation reaction using different catalyst: The conversion of ethylbenzene hydroperoxide X_{EBHP}, the molar selectivity to secondary product acetophenone ACP, S_{ACP}, and the pseudo-first order kinetic rate constant for the heterogeneous reaction, k′.

The conversion of ethylbenzene hydroperoxide is defined as the percentage of ethylbenzene hydroperoxide reacted. The selectivity to acetophenone ACP is defined as the molar percentage of produced ACP with respect to reacted EBHP. The kinetic rate constant k′ (L/g·h) is defined by the following formula:

$${k}^{′} = \frac{{F}_{AO}}{{C}_{AO}} \cdot \frac{1}{w} ln ( \frac{1}{1 - {X}_{A}} )$$

wherein F_{A0} is the rate (mol/h) of EBHP at the reactor inlet, C_{A0} is the concentration (mol/L) of EBHP at the reactor inlet, w represents the catalyst weight (grams), and X_{A} is the conversion of EBHP as defined above. The calculations were made considering a density of the reactant mixture at 100° C. of 509 g/L.

Testing results are summarized in Table 2.

Due to its unique properties, the catalyst prepared in this example shows very high conversion X_{EBHP} selectivity and kinetic rate constant k′, furthermore, at the same time the selectivity to the undesired product acetophenone S_{ACP} is low.

### Example 2

Catalyst 2 was prepared and characterized as described in example 1 except for the drying step. The as-synthesized precursor (Xwa=55.5 wt %) was placed into a pre-heated drying cabinet at 160° C. and dried keeping this temperature for 17 h. The dried material had a residual water content, Xwb, of Xwb=3.0 wt %.

Materials obtained were characterized with respect to their physico-chemical properties and tested with respect to their catalytic properties as described in example 1. Results are summarized in tables 1 and 2.

### Example 3: (Comparative-Ti-Content Too Low)

Catalyst 3 was prepared and characterized as described in example 1 except for the Ti-content of the as-synthesized hydrogel (with the hydrogel possessing an Xwa of Xwa=58 wt %), the ageing time and temperature and the calcination step. The Ti-content was 0.61 wt % (TiO₂ 1.02 wt %). The ageing solution was cycled over the fixed bed gel particles in the reactor for 5-6 h at 80-85° C.

The dried material (possessing a residual water content, Xwb, of Xwb=2.9 wt %) was calcined in a furnace under static conditions for 2 h at 600° C. (applying a heating rate of 5° C./min to reach the desired temperature).

Materials obtained were characterized with respect to their physico-chemical properties and tested with respect to their catalytic properties as described in example 1. Results are summarized in tables 1 and 2.

In comparison to catalysts 1 and 2, catalyst 3 has a lower Ti-content, a lower proportion of Ti-centers that are pentahedrally coordinated, exhibits a lower conversion X_{EBHP} selectivity and much lower kinetic rate constant k′. In addition, the selectivity to the undesired product S_{ACP} is much higher for catalyst 3.

### Example 4: (Comparative-Ti-Content Too High)

Catalyst 4 was prepared and characterized as described in example 1 except for the Ti-content of the as-synthesized hydrogel (with the hydrogel possessing an Xwa of Xwa=57 wt % prior to drying) the ageing time and temperature and the calcination step. The Ti-content was 2.04 wt % (TiO₂ 3.41 wt %). The ageing solution was cycled over the fixed bed gel particles in the reactor for 5-6 h at 80-85° C. The dried material, exhibiting a residual water content, Xwb, of Xwb=3.5 wt %, was calcined in a furnace under static conditions for 2 h at 600° C. (applying a heating rate of 5° C./min to reach the desired temperature).

Materials obtained were characterized with respect to their physico-chemical properties and tested with respect to their catalytic properties as described in example 1. Results are summarized in tables 1 and 2.

### Example 5: (not Hydrophobized)

Catalyst 5 was prepared and characterized as described in example 1 (with the as synthesized hydrogel possessing an Xwa of Xwa=55.5 wt % and a Ti-content of 1.69 wt % (2.82 wt % TiO₂) as well as a residual water content, Xwb, after drying of Xwb=3.2 wt %) with the exception that the calcined material was not hydrophobized. The material tested was used in the calcined form.

Materials obtained were characterized with respect to their physico-chemical properties and tested with respect to their catalytic properties as described in example 1. Results are summarized in tables 1 and 2.

### Example 6: (Comparative-Catalyst Prepared by Gas Phase Deposition of TiCl₄ on a Silica Support)

Catalyst 6 was prepared by gas phase deposition of titaniumtetrachloride (TiCl₄) on a dry silica support. In a first pre-treatment step, 120 g silica (Aerolyst 3041, A_{S,BET}=179 m²/g, V_{P,total}=1.05 cm³/g, D_{P,BJH}=19 nm) were treated with aqueous HCl (w=4 mol/l) at 70° C. for 60 min, followed by filtration and drying of the solid at 110° C. for 4 h. The pre-treated silica was then brought into contact with a TiCl₄ containing gas stream, obtained by heating TiCl₄ to 200° C. using a flow of nitrogen N₂ (60 l/h) as carrier gas. The impregnated silica (titanium content of 1.8 wt %) was subsequently calcined at 590° C. for 6 h, followed by a steam treatment at 220° C. for 3 h. Finally, the steamed material was hydrophobized as described in Example 1 using 8 g of steamed material, 4 g hexamethyldisilazane and 52 ml toluene.

Materials obtained were characterized with respect to their physico-chemical properties and tested with respect to their catalytic properties as described in example 1. Results are summarized in tables 1 and 2.

### Example 7: (Ageing Step Prior to Ti-Impregnation)

1800 liter of diluted sulfuric acid (16 wt %) were filled in a fixed-bed reactor. Then 630 l of water glass (viscosity of 500 mPa*s) were prilled into the diluted sulfuric acid. The water glass drops gelled instantly after contact with the acid and formed gel particles. The gel particles sank down and formed a bed of gel particles in the reactor.

The sulfuric acid was circulated over the bed of gel particles in the reactor and an ion exchange column with a flow rate of 1600-2000 l per h. The acid was intended to remove three, four and five valence metal ion impurities from the gel particles and transport them onto the ion exchange column where they are adsorbed. The cycling was continued for approximately 75-80 min.

Then the acid was replaced with fresh diluted sulfuric acid. The acid was heated to 60° C. During heating the acid was cycled over the bed and the ion exchange column again. Cycling was performed at a flow rate of 4000 l per hour for 80 min.

Then the acid was discharged, and the gel particles were rinsed with deionized water for 60 min with a flow rate of 8000 l per h.

The solution was replaced with deionized water and the gel particles were washed with deionized water for with a flow rate of 10000 l per h. 80 l of a solution of ammonium acetate (50 wt %) were dosed into the stream of deionized water, thereby ammonium acetate concentration was adjusted to approx. 7.5 wt % and the pH value was raised to 5.5-6.5. The ageing solution was cycled over the fixed bed gel particles in the reactor for 5-6 h at 80-85° C.

A solution of sulfuric acid (25 wt %) and titanium sulfate (3-4 wt %, aiming at a titanium content of the catalyst hydrogel precursor of 1.7 wt %) was cycled over the fixed bed gel particles in the reactor for 120 min.

The sulfuric acid solution was replaced by deionized water and washed with deionized water for 60 minutes with a flow rate of 8000 l per h.

At the end, the water was rinsed off and the titanium doped silica hydrogel was discharged from the reactor. The as synthesized hydrogel possesses a residual water content, Xwa, of approximately Xwa=57.5 wt % and a Ti-content of 1.65 wt % (2.75 wt % TiO₂). The titanium content of the samples was determined by elemental analysis via optical emission spectrometry with inductively coupled plasma (ICP-OES) as described in example 1. Ti-contents of solid materials referred to in the present invention have been calculated on a dry-mass basis.

500 g of the hydrogel that had been obtained was then transferred to a drying cabinet, heated up to 160° C. applying a heating rate of 3° C./min and kept at this temperature for 15 h in air atmosphere. For the resulting material a residual water content, Xwb, of Xwb=3.1 wt % was found.

Thereafter, the dried material was calcined in a furnace under static conditions for 2 h at 800° C. (applying a heating rate of 5° C./min to reach the desired temperature).

Materials were characterized by share of intensity below 275 nm SI_{b275} and position of band maximum (highest point of curve) as described in example 1. Results are summarized in table 1.

Calcination was followed by a hydrophobization step. For this purpose, 40 g of the calcined material were placed in a 3-necked round bottom flask followed by the addition of 264 ml toluene and 20 g of hexadimethylsilazane. The reaction mixture was smoothly mixed (100 min⁻¹) for 1 hour at 25° C., subsequently heated to 110° C. and kept at this temperature for 3 hours. After cooling down to 25° C. the solid was filtered off, partially dried in a flow of nitrogen and placed into a vacuum dryer at a temperature not exceeding 110° C. overnight in order to remove the solvent.

Materials obtained were characterized with respect to their physico-chemical properties as described in example 1. Results are summarized in table 1.

### Evaluation of Catalyst Performance:

In order to evaluate performance of the catalyst, a continuous fixed bed epoxidation of propene with ethylbenzene hydroperoxide was carried out. Performance was tested in an isothermal fixed bed reactor (diameter=10 mm, length=1.75 m) and the temperature was controlled by a jacket with thermal oil. The catalyst loading is indicated in Table 2, depending on the test. The catalyst was mixed with glass balls (1 mm) to improve temperature control and both top and bottom had glass balls (3 mm) with a bigger diameter to protect the catalytic bed. The mixture feed was fed to the reactor from top to bottom

Epoxidation of propene was carried out with a solution of ethylbenzene hydroperoxide. A solution of 35 wt % ethylbenzene hydroperoxide in ethylbenzene, made by oxidation of ethylbenzene with air, was used. The solution was pretreated according to the procedure described in U.S. Pat. No. 5,883,268. After pretreatment, the ethylbenzene hydroperoxide solution had an acids content below 500 ppm, a water content below 5000 ppm, and it contained also some methyl phenyl ketone and/or 1-phenyl-ethanol formed in the preceding oxidation. A typical ethylbenzene hydroperoxide feed comprised 33-36 wt % ethylbenzene hydroperoxide, 53-54 wt % ethylbenzene, 2-4 wt % 1-phenyl-ethanol and 3-5 wt % methyl phenyl ketone. The propene was added to the ethylbenzene hydroperoxide feed, and the mixture was fed into the fixed bed reactor.

Ethylbenzene hydroperoxide (EBHP) solution and propene were fed in continuous in the fixed bed reactor at 25° C. Amounts of EBHP and propene were adjusted with amount of catalyst keeping the ratio between EBHP, propene and catalyst constant. For exact amounts see Table 2.

The reactor temperature was controlled at 100° C., and the reaction was carried out at a pressure of 60 bar. Once the reactor was in a continuous operation at the set temperature, samples were taken every 24 h to analyze the outlet composition by GC and HPLC, using Agilent 6850 and Agilent 1100 Series chromatographs.

In Table 2, the following results are summarized for the epoxidation reaction using different catalyst: The conversion of ethylbenzene hydroperoxide X_{EBHP}, the molar selectivity to secondary product acetophenone ACP, S_{ACP}, and the pseudo-first order kinetic rate constant for the heterogeneous reaction, k′.

The conversion of ethylbenzene hydroperoxide is defined as the percentage of ethylbenzene hydroperoxide reacted. The selectivity to acetophenone ACP is defined as the molar percentage of produced ACP with respect to reacted EBHP. The kinetic rate constant k′ (L/g·h) is defined by the following formula:

$${k}^{′} = \frac{{F}_{AO}}{{C}_{AO}} \cdot \frac{1}{w} ln ( \frac{1}{1 - {X}_{A}} )$$

wherein F_{A0} is the rate (mol/h) of EBHP at the reactor inlet, C_{A0} is the concentration (mol/L) of EBHP at the reactor inlet, w represents the catalyst weight (grams), and X_{A} is the conversion of EBHP as defined above. The calculations were made considering a density of the reactant mixture at 100° C. of 509 g/L.

Testing results are summarized in Table 2.

### Example 8: (Comparative-Xwb Too Low)

1800 liter of diluted sulfuric acid (16 wt %) were filled in a fixed-bed reactor. Then 630 l of water glass (viscosity of 500 mPa*s) were prilled into the diluted sulfuric acid. The water glass drops gelled instantly after contact with the acid and formed gel particles. The gel particles sank down and formed a bed of gel particles in the reactor.

The sulfuric acid was circulated over the bed of gel particles in the reactor and an ion exchange column with a flow rate of 1600-2000 l per h. The acid was intended to remove three, four and five valence metal ion impurities from the gel particles and transport them onto the ion exchange column where they are adsorbed. The cycling was continued for approximately 75-80 min.

Then the acid was replaced with fresh diluted sulfuric acid. The acid was heated to 60° C. During heating the acid was cycled over the bed and the ion exchange column again. Cycling was performed at a flow rate of 4000 l per hour for 80 min.

Then the acid was discharged, and the gel particles were rinsed with deionized water for 60 min with a flow rate of 8000 l per h.

The solution was replaced with deionized water and the gel particles were washed with deionized water with a flow rate of 10000 l per h. 80 l of a solution of ammonium acetate (50 wt %) were dosed into the stream of deionized water, thereby ammonium acetate concentration was adjusted to approx. 7.5 wt % and the pH value to was raised to 5.5-6.5. The ageing solution was cycled over the fixed bed gel particles in the reactor for 5-6 h at 80-85° C.

Afterwards, the ammonium acetate solution was replaced with deionized water and washed with deionized water for 60 minutes with a flow rate of 8000 l per h.

At the end, the water was rinsed off and the silica hydrogel was discharged from the reactor. The as synthesized hydrogel possesses a residual water content, Xwa, of approximately Xwa=58 wt %.

500 g of the hydrogel that had been obtained were then transferred to a drying cabinet, heated up to 160° C. applying a heating rate of 3° C./min and kept at this temperature for 18 h in air atmosphere. For the resulting material a residual water content, Xwb, of Xwb=0.16 wt % was found.

Successively, the dried hydrogel was then impregnated with a diluted titanium sulfate solution (15 m % titanium sulfate solution diluted in water to fill the pore volume of the hydrogel, aiming at a titanium content of the catalyst hydrogel precursor of 1.7 wt %)

The impregnated gel that had been obtained was then transferred to a drying cabinet, heated up to 160° C. applying a heating rate of 3° C./min and kept at this temperature for 15 h in air atmosphere.

Thereafter, the dried material was calcined in a furnace under static conditions for 2 h at 800° C. (applying a heating rate of 5° C./min to reach the desired temperature).

Materials obtained were characterized with respect to their physico-chemical properties as described in example 1. Results are summarized in table 1.

### Evaluation of Catalyst Performance:

In order to evaluate performance of the catalyst, a continuous fixed bed epoxidation of propene with ethylbenzene hydroperoxide was carried out. Performance was tested in an isothermal fixed bed reactor (diameter=10 mm, length=1.75 m) and the temperature was controlled by a jacket with thermal oil. The catalyst loading is indicated in Table 2, depending on the test. The catalyst was mixed with glass balls (1 mm) to improve temperature control and both top and bottom had glass balls (3 mm) with a bigger diameter to protect the catalytic bed. The mixture feed was fed to the reactor from top to bottom

Epoxidation of propene was carried out with a solution of ethylbenzene hydroperoxide. A solution of 35 wt % ethylbenzene hydroperoxide in ethylbenzene, made by oxidation of ethylbenzene with air, was used. The solution was pretreated according to the procedure described in U.S. Pat. No. 5,883,268. After pretreatment, the ethylbenzene hydroperoxide solution had an acids content below 500 ppm, a water content below 5000 ppm, and it contained also some methyl phenyl ketone and/or 1-phenyl-ethanol formed in the preceding oxidation. A typical ethylbenzene hydroperoxide feed comprised 33-36 wt % ethylbenzene hydroperoxide, 53-54 wt % ethylbenzene, 2-4 wt % 1-phenyl-ethanol and 3-5 wt % methyl phenyl ketone. The propene was added to the ethylbenzene hydroperoxide feed, and the mixture was fed into the fixed bed reactor.

Ethylbenzene hydroperoxide (EBHP) solution and propene were fed in continuous in the fixed bed reactor at 25° C. Amounts of EBHP and propene were adjusted with amount of catalyst keeping the ratio between EBHP, propene and catalyst constant. For exact amounts see Table 2.

The reactor temperature was controlled at 100° C., and the reaction was carried out at a pressure of 60 bar. Once the reactor was in a continuous operation at the set temperature, samples were taken every 24 h to analyze the outlet composition by GC and HPLC, using Agilent 6850 and Agilent 1100 Series chromatographs.

In Table 2, the following results are summarized for the epoxidation reaction using different catalyst: The conversion of ethylbenzene hydroperoxide X_{EBHP}, the molar selectivity to secondary product acetophenone ACP, S_{ACP}, and the pseudo-first order kinetic rate constant for the heterogeneous reaction, k′.

The conversion of ethylbenzene hydroperoxide is defined as the percentage of ethylbenzene hydroperoxide reacted. The selectivity to acetophenone ACP is defined as the molar percentage of produced ACP with respect to reacted EBHP. The kinetic rate constant k′ (L/g·h) is defined by the following formula:

$${k}^{′} = \frac{{F}_{AO}}{{C}_{AO}} \cdot \frac{1}{w} ln ( \frac{1}{1 - {X}_{A}} )$$

wherein F_{A0} is the rate (mol/h) of EBHP at the reactor inlet, C_{A0} is the concentration (mol/L) of EBHP at the reactor inlet, w represents the catalyst weight (grams), and X_{A} is the conversion of EBHP as defined above. The calculations were made considering a density of the reactant mixture at 100° C. of 509 g/L.

Testing results are summarized in Table 2.

### Example 9: (Comparative-Xwb Too High)

1800 liter of diluted sulfuric acid (16 wt %) were filled in a fixed-bed reactor. Then 630 l of water glass (viscosity of 500 mPa*s) were prilled into the diluted sulfuric acid. The water glass drops gelled instantly after contact with the acid and formed gel particles. The gel particles sank down and formed a bed of gel particles in the reactor.

The sulfuric acid was circulated over the bed of gel particles in the reactor and an ion exchange column with a flow rate of 1600-2000 l per h. The acid was intended to remove three, four and five valence metal ion impurities from the gel particles and transport them onto the ion exchange column where they are adsorbed. The cycling was continued for approximately 75-80 min.

Then the acid was replaced with fresh diluted sulfuric acid. The acid was heated to 60° C. During heating the acid was again cycled over the bed and the ion exchange column. Cycling was performed at a flow rate of 4000 l per hour for 80 min.

Then the acid was discharged, and the gel particles were rinsed with deionized water for 60 min with a flow rate of 8000 l per h.

The solution was replaced with deionized water and the gel particles were washed with deionized water for with a flow rate of 10000 l per h. 80 l of a solution of ammonium acetate (50 wt %) were dosed into the stream of deionized water, thereby ammonium acetate concentration was adjusted to approx. 7.5 wt % and the pH value to was raised to 5.5-6.5. The ageing solution was cycled over the fixed bed gel particles in the reactor for 5-6 h at 80-85° C.

Afterwards, the ammonium acetate solution was replaced by deionized water and washed with deionized water for 60 minutes with a flow rate of 8000 l per h.

At the end, the water was rinsed off and the silica hydrogel was discharged from the reactor. The as synthesized hydrogel possesses a residual water content, Xwa, of approximately Xwa=58 wt %.

500 g of the hydrogel that had been obtained was then transferred to a drying cabinet, heated up to 160° C. applying a heating rate of 3° C./min and kept at this temperature for 1 h in air atmosphere. For the resulting material a residual water content, Xwb, of Xwb=25 wt % was found.

Successively, the dried gel was then impregnated with a diluted titanium sulfate solution (15 m % titanium sulfate solution diluted in water to fill the pore volume of the hydrogel, aiming at a titanium content of the catalyst hydrogel precursor of 1.7 wt %)

The impregnated gel that had been obtained was then transferred to a drying cabinet, heated up to 160° C. applying a heating rate of 3° C./min and kept at this temperature for 15 h in air atmosphere.

Thereafter, the dried material was calcined in a furnace under static conditions for 2 h at 800° C. (applying a heating rate of 5° C./min to reach the desired temperature).

Materials obtained were characterized with respect to their physico-chemical properties as described in example 1. Results are summarized in table 1.

### Evaluation of Catalyst Performance:

In order to evaluate performance of the catalyst, a continuous fixed bed epoxidation of propene with ethylbenzene hydroperoxide was carried out. Performance was tested in an isothermal fixed bed reactor (diameter=10 mm, length=1.75 m) and the temperature was controlled by a jacket with thermal oil. The catalyst loading is indicated in Table 2, depending on the test. The catalyst was mixed with glass balls (1 mm) to improve temperature control and both top and bottom had glass balls (3 mm) with a bigger diameter to protect the catalytic bed. The mixture feed was fed to the reactor from top to bottom

Epoxidation of propene was carried out with a solution of ethylbenzene hydroperoxide. A solution of 35 wt % ethylbenzene hydroperoxide in ethylbenzene, made by oxidation of ethylbenzene with air, was used. The solution was pretreated according to the procedure described in U.S. Pat. No. 5,883,268. After pretreatment, the ethylbenzene hydroperoxide solution had an acids content below 500 ppm, a water content below 5000 ppm, and it contained also some methyl phenyl ketone and/or 1-phenyl-ethanol formed in the preceding oxidation. A typical ethylbenzene hydroperoxide feed comprised 33-36 wt % ethylbenzene hydroperoxide, 53-54 wt % ethylbenzene, 2-4 wt % 1-phenyl-ethanol and 3-5 wt % methyl phenyl ketone. The propene was added to the ethylbenzene hydroperoxide feed, and the mixture was fed into the fixed bed reactor.

Ethylbenzene hydroperoxide (EBHP) solution and propene were fed in continuous in the fixed bed reactor at 25° C. Amounts of EBHP and propene were adjusted with amount of catalyst keeping the ratio between EBHP, propene and catalyst constant. For exact amounts see Table 2.

The reactor temperature was controlled at 100° C., and the reaction was carried out at a pressure of 60 bar. Once the reactor was in a continuous operation at the set temperature, samples were taken every 24 h to analyze the outlet composition by GC and HPLC, using Agilent 6850 and Agilent 1100 Series chromatographs.

In Table 2, the following results are summarized for the epoxidation reaction using different catalyst: The conversion of ethylbenzene hydroperoxide X_{EBHP}, the molar selectivity to secondary product acetophenone ACP, S_{ACP}, and the pseudo-first order kinetic rate constant for the heterogeneous reaction, k″.

The conversion of ethylbenzene hydroperoxide is defined as the percentage of ethylbenzene hydroperoxide reacted. The selectivity to acetophenone ACP is defined as the molar percentage of produced ACP with respect to reacted EBHP. The kinetic rate constant k′ (L/g·h) is defined by the following formula:

$${k}^{′} = \frac{{F}_{AO}}{{C}_{AO}} \cdot \frac{1}{w} ln ( \frac{1}{1 - {X}_{A}} )$$

wherein F_{A0} is the rate (mol/h) of EBHP at the reactor inlet, C_{A0} is the concentration (mol/L) of EBHP at the reactor inlet, w represents the catalyst weight (grams), and X_{A} is the conversion of EBHP as defined above. The calculations were made considering a density of the reactant mixture at 100° C. of 509 g/L.

Testing results are summarized in Table 2.

TABLE 1
Physico-chemical properties, i.e. specific BET surface area A_{S, BET}, average
pore-width D_{P, BJH}, total pore-volume V_{P, total,} share of intensity below
275 nm Sl_{b275} and band maximum as derived from DR-UV/Vis as well as
Ti-content for catalysts obtained as described in example 1-9
Physico-chemical properties

| | | | | | | Band | |
|---|---|---|---|---|---|---|---|
| | | A¹_{S, BET} | D²_{P, BJH} | V³_{P, total} | Sl_{b275}⁴ | maximum⁵ | Ti⁶-content |
| Example | | [m²/g] | [nm] | [cm³/g] | [%] | [nm] | [wt %] |
| 1 | Calcined | 491 | 5.3 | 0.8 | 90 ± 3 | 233 ± 5 | 1.66 |
| | material | | | | | | |
| | Hydrophobized | 409 | 4.8 | 0.7 | 95 ± 3 | 226 ± 5 | |
| | material | | | | | | |
| 2 | Calcined | 491.6 | 5.2 | 0.8 | 87 ± 3 | 237 ± 5 | 1.66 |
| | material | | | | | | |
| | Hydrophobized | 416.2 | 4.8 | 0.7 | 90 ± 3 | 222 ± 5 | |
| | material | | | | | | |
| 3 | Calcined | 487 | 5.2 | 0.75 | 80 ± 3 | 240 ± 5 | 0.61 |
| | material | | | | | | |
| | Hydrophobized | | | | | 233 ± 5 | |
| | material | | | | | | |
| 4 | Calcined | 489 | 4.9 | 0.0.73 | 75 ± 3 | 245 ± 5 | 2.04 |
| | material | | | | | | |
| | Hydrophobized | | | | 80 ± 3 | 235 ± 5 | |
| | material | | | | | | |
| 5 | Calcined | 491 | 5.3 | 0.8 | 90 ± 3 | 233 ± 5 | 1.66 |
| | material | | | | | | |
| 6 | Hydrophobized | 173 | 19 | 0.97 | 60 ± 3 | 255 ± 5 | 1.80 |
| | material | | | | | | |
| 7 | Hydrophobized | 490 | 5.3 | 0.8 | 90 ± 3 | 232 ± 5 | 1.65 |
| | material | | | | | | |
| 8 | Calcined | 400 | 8.9 | 0.8 | 54 ± 3 | 270 ± 5 | 1.54 |
| | material | | | | | | |
| 9 | Calcined | 400 | 8.9 | 0.8 | 56 ± 3 | 265 ± 5 | 1.96 |
| | material | | | | | | |

¹specific BET surface area A_{S, BET}/
²average pore-width D_{P, BJH}/
³total pore-volume V_{P, total} derived from nitrogen physisorption at 77K;
⁴⁵as derived from DR-UV/Vis, measured reflection was calculated into Kubelka-Munk-units;
⁶determined by elemental analysis via ICP-OES. Experimental procedures described in example 1.

TABLE 2
Catalytic performance of the catalysts tested, i.e. ethylbenzene
hydroperoxide X_{EBHP}, selectivity to acetophenone S_{ACP} and kinetic rate
constant k′ values. Testing conditions are indicated in the footnote.
Catalytic performance

| | | X_{EBHP} [%] | | | S_{ACP} [%] | | | k′ [L/g · h] | | |
|---|---|---|---|---|---|---|---|---|---|---|
| Example | | 300 h | 400 h | 1500 h | 300 h | 400 h | 1500 h | 300 h | 400 h | 1500 h |
| 1* | Hydrophobized | 99 | 98 | 87 | 2.1 | 2.3 | 2.4 | 0.166 | 0.142 | 0.073 |
| | material | | | | | | | | | |
| 2* | Hydrophobized | 98 | 97 | n.d. | 2.3 | 2.0 | n.d. | 0.146 | 0.134 | n.d. |
| | material | | | | | | | | | |
| 3** | Hydrophobized | 33 | 31 | n.d. | n.d. | 3.7 | n.d. | 0.014 | 0.013 | n.d. |
| | material | | | | | | | | | |
| 4** | Hydrophobized | 54 | 49 | n.d. | 1.6 | 2.1 | n.d. | 0.028 | 0.025 | n.d. |
| | material | | | | | | | | | |
| 5* | Calcined material | 90 | 87 | n.d. | 3.8 | 3.6 | n.d. | 0.086 | 0.076 | n.d. |
| 6** | Hydrophobized | 86 | 82 | n.d. | 2.1 | 2.9 | n.d. | 0.073 | 0.062 | n.d. |
| | material | | | | | | | | | |
| 7* | Hydrophobized | 99 | 98 | 87 | 2.1 | 2.2 | 2.4 | 0.163 | 0.141 | 0.074 |
| | material | | | | | | | | | |
| 8* | calcined material | 85 | n.d. | n.d. | 4.3 | n.d. | n.d. | 0.070 | n.d. | n.d. |
| 9* | calcined material | 84 | n.d. | n.d. | 4.1 | n.d. | n.d. | 0.067 | n.d. | n.d. |

*80 g/h of ethylbenzene hydroperoxide solution/256 g/h of propene @ 25° C./the reactor was charged with 20 g of catalyst.
**40 g/h of ethylbenzene hydroperoxide solution/128 g/h of propene @ 25° C./reactor was charged with 10 g of catalyst
n.d. not determined

As can be seen from Table 2 above, catalysts according to the invention show a higher conversion of EBHP X_{EBHP} and higher kinetic rate constants k′ than comparative ones, along the different TOS measurements. It is important to mention that selectivity to acetophenone, S_{ACP}, is a critical parameter, as the percentage of ACP constitutes process losses. The catalysts according to the present invention show lower values of selectivity towards the secondary product ACP at a high conversion of EBHP X_{EBHP} and kinetic rate constants k′ and therefore significantly exceed the performance of the comparative ones

1-10. (canceled)

11. A process for the manufacture of catalysts comprising amorphous titanium doped silica with pentahedrally coordinated titanium species, said process comprising the following steps:
a) sol-gel-synthesis of a catalyst hydrogel-precursor, comprising the steps:
i) adding water glass exhibiting a viscosity in the range of 400 to 600 mPa*s into an acid, thereby yielding silica gel exhibiting a residual water content, Xwa, in the range between Xw1 and Xw2;
ii) removing impurities from said silica gel by washing the silica gel with an acidic aqueous solution, thereby yielding purified silica gel;
iii) forming a catalyst hydrogel-precursor by either alternative a) or b) as follows:
alternative a):
doping said purified silica gel by contacting it with an acidic aqueous solution of a titanium salt, thereby yielding titanium-doped silica gel; and
aging said titanium-doped silica gel by suspending the titanium-doped silica gel in an aging-solution for between 4 h and 6 h at a temperature between 60° C. and 90° C., wherein the liquid part of the suspension formed thereby exhibits a pH value of 5.5 to 7.5; thereby yielding the catalyst hydrogel-precursor having a titanium content, Xt, in the range between Xt1 and Xt2;
alternative b):
aging said purified silica gel by suspending it in an aging solution for between 4 h and 6 h at between 60° C. and 90° C., wherein the liquid part of the suspension formed thereby exhibits a pH value in the range of 5.5 to 7.5; thereby yielding aged, purified silica gel; and
doping said aged, purified silica gel by contacting it with an acidic aqueous solution of a titanium salt; thereby yielding the catalyst hydrogel-precursor having a titanium content, Xt. in the range between Xt1 and Xt2;
b) drying the catalyst hydrogel precursor to a residual water content, Xwb, of the dried catalyst hydrogel-precursor in a range between Xw3 and Xw4;
c) calcining the dried catalyst hydrogel-precursor;
wherein:
Xw1=40 wt %, Xw2=60 wt %;
Xt1=0.85 wt %, Xt2=1.9 wt %;
Xw3=1 wt %, Xw4=7 wt %;
the residual water content, Xwa and Xwb, is determined as the weight loss exhibited by a 100 g aliquot after drying at 200° C. for four hours in a drying cabinet under atmospheric conditions;
the titanium content, Xt, of samples is determined by elemental analysis via optical emission spectrometry with inductively coupled plasma (ICP-OES); and
the Ti content of solid materials is calculated on a dry-mass basis and corresponds to the content of elemental titanium.

12. The process of claim 11, further comprising:
d) hydrophobizing the calcined catalyst hydrogel-precursor with a hydrophobizing agent in liquid phase.

13. The process of claim 11, wherein Xt is selected in the range between Xt1=1 wt % and Xt2=1.9 wt %.

14. The process of claim 12, wherein Xt is selected in the range between Xt1=1 wt % and Xt2=1.9 wt %.

15. The process of claim 11, wherein Xt is selected in the range between Xt1=1.4 wt % and Xt2=1.8 wt %.

16. The process of claim 11, wherein the catalyst hydrogel-precursor exhibits a residual water content Xwa in the range between Xw1=45 wt % and Xw2=60 wt %.

17. The process of claim 14, wherein the catalyst hydrogel-precursor exhibits a residual water content Xwa in the range between Xw1=45 wt % and Xw2=60 wt %.

18. The process of claim 11, wherein the catalyst hydrogel-precursor exhibits a residual water content Xwa in the range between Xw1=52 wt % and Xw2=57 wt %.

19. A catalyst for transforming propylene into propylene oxide in the propylene oxide/styrene monomer-process, made by the process of claim 11.

20. The catalyst of claim 19, wherein said catalyst comprises a specific BET surface area As. BET in the range between 350 and 650 m²/g.

21. The catalyst of claim 19, said catalyst comprising a specific BET surface area As. BET in the range between 400 and 550 m²/g.

22. The catalyst of claim 19, comprising an average pore width, D_{P,BJH}, in the range between 4 and 8 nm.

23. The catalyst of claim 20, comprising an average pore width, D_{P,BJH}, in the range between 4 and 8 nm.

24. The catalyst of claim 19, comprising an average pore-width, D_{P,BJH}, in the range between 4 and 5 nm.

25. The catalyst of claim 19, comprising a pore-volume, V_{P total}, in the range between 0.4 and 1.0 cm³/g.

26. The catalyst of claim 23, comprising a pore-volume, V_{P, total}, in the range between 0.4 and 1.0 cm³/g.

27. The catalyst of claim 19, comprising a pore-volume, V_{P, total}, in the range between 0.7 to 0.9 cm³/g.

28. The catalyst of claim 19, comprising the following characteristics:
a) a titanium content in the range between 1.6 and 1.8 wt %;
b) a specific BET surface area, As, BET, between 400 and 450 m²/g;
c) an average pore-width, D_{P, BJH}, between 4.5 and 5 nm;
d) a pore volume, V_{P, total}, between 0.7 and 0.8 cm³/g.

29. The catalyst of claim 28, wherein:
a) the titanium content, Xt, of samples is determined by elemental analysis via optical emission spectrometry;
b) the Ti content of solid materials is calculated on a dry mass basis and corresponds to the content of elemental titanium;
c) the average pore width, D_{P,BJH}, is derived from nitrogen physisorption isotherms; and
d) the pore volume, V_{P,total}, is derived from nitrogen physisorption isotherms.

30. A process for the preparation of propylene oxide comprising reacting propene with ethylbenzene hydroperoxide in the presence of the catalyst of claim 19.
